(19) United States
(12) Patent Application Publication
(10) Pub. No.: US 2022/0241280 A1
(43) Pub. Date: Aug. 4, 2022

(54) PHARMACEUTICAL COMPOSITION AND THERAPEUTIC METHOD FOR TREATING FGFR1 VARIANT-POSITIVE BRAIN TUMOR

(71) Applicant: TAIHO PHARMACEUTICAL CO., LTD., Tokyo (JP)

(72) Inventors: Hiroshi HIRAI, Tokyo (JP); Akihiro MIURA, Tsukuba-shi, Ibaraki (JP)

(73) Assignee: TAIHO PHARMACEUTICAL CO., LTD., Tokyo (JP)

(21) Appl. No.: 17/432,158

(22) PCT Filed: Feb. 19, 2020
(86) PCT No.: PCT/JP2020/006464

§ 371 Date: Aug. 19, 2021

(30) Foreign Application Priority Data
Feb. 20, 2019 (JP) PCT/JP2019/006265

(51) Int. Cl. A61K 31/519; A61K 9/08

(52) U.S. Cl. CPC A61K 31/519; A61K 9/08

(57) ABSTRACT

The present invention provides a pharmaceutical composition for treating a patient with an FGFR1 mutant-positive brain tumor, the composition comprising (S)-1-(3-(4-amino-3-((3,5-dimethoxyphenyl)ethynyl)-1H-pyrazolo[3,4-d]pyrimidin-1-yl)pyrrolidin-1-yl)prop-2-en-1-one or a pharmaceutically acceptable salt thereof as an active ingredient; and a therapeutic method using the pharmaceutical composition.

### TECHNICAL FIELD

The present invention relates to a pharmaceutical composition and therapeutic method for treating an FGFR1 mutant-positive brain tumor.

### BACKGROUND ART

Fibroblast growth factors (FGFs) are expressed in various tissues, and are one of the growth factors that regulate cell proliferation and differentiation. The physiological activity of the FGFs is mediated by fibroblast growth factor receptors (FGFRs), which are specific cell surface receptors. FGFRs belong to a receptor protein tyrosine kinase family, and comprise an extracellular ligand-binding domain, a single transmembrane domain, and an intracellular tyrosine kinase domain. Four types of FGFRs (FGFR1, FGFR2, FGFR3, and FGFR4) have been heretofore identified. FGFRs bind to FGFs to form dimers, and are activated by phosphorylation. Activation of the receptors induces mobilization and activation of specific downstream signal transduction molecules, thereby developing physiological functions.

Some reports have been made about the relationship between aberrant FGF/FGFR signaling and diseases related to tumors in humans. Aberrant activation of FGF/FGFR signaling in human tumors is considered to be attributable to overexpression of FGFRs and/or gene amplification, gene mutation, chromosomal translocation, insertion and inversion, gene fusion, or an autocrine or paracrine mechanism by overproduction of FGFs (ligands) (NPL 1, 2, and 3).

Non-patent Literature 4 and 5 reported single-amino-acid substitution mutations such as N546K, K656E, K656D, K656N, and K656M in FGFR1, or a transforming acidic coiled-coil containing protein 1 (TACC1) fusion in brain tumor; and they suggest that these genetic mutations may be a driving force of canceration.

Patent Literature 1 reported about disubstituted benzene alkynyl compounds having an FGFR inhibitory effect. Patent Literature 2 and 3 respectively reported that these compounds are effective against a tumor with a specific FGFR2 mutation, and that an intermittent administration schedule may be effective.

### CITATION LIST

### Patent Literature

PTL 1: WO2013/108809
PTL 2: WO2015/008844
PTL 3: WO2015/008839

### Non-Patent Literature

NPL 1: J. Clin. Oncol., 24, 3664-3671 (2006)
NPL 2: Mol. Cancer Res., 3 (12), 655-667 (2005)
NPL 3: Cancer Res., 70 (5), 2085-2094 (2010)
NPL 4: Nat. Genet., 45 (8), 927-932 (2013)
NPL 5: Science, 337 (6099), 1231-1235 (2012)

### SUMMARY OF INVENTION

### Technical Problem

The present invention aims to provide a pharmaceutical composition for treating a brain tumor with an FGFR1 mutation, and a therapeutic method using the pharmaceutical composition.

### Solution to Problem

As a result of extensive research to attain the above object, the present inventors found that (S)-1-(3-(4-amino-3-((3,5-dimethoxyphenyl)ethynyl)-1H-pyrazolo[3,4-d]pyrimidin-1-yl)pyrrolidin-1-yl)prop-2-en-1-one inhibits phosphorylation of mutant FGFR1, and has an excellent anti-tumor effect against a brain tumor having an FGFR1 mutation.

Specifically, the present invention includes the following [1] to [23].

[1] A pharmaceutical composition for treating an FGFR1 mutant-positive brain tumor patient, comprising (S)-1-(3-(4-amino-3-((3,5-dimethoxyphenyl)ethynyl)-1H-pyrazolo[3,4-d]pyrimidin-1-yl)pyrrolidin-1-yl)prop-2-en-1-one or a pharmaceutically acceptable salt thereof as an active ingredient.
[2] The pharmaceutical composition according to Item 1, wherein the brain tumor patient has a mutation in which the 546^{th} asparagine of FGFR1 is substituted with another amino acid.
[3] The pharmaceutical composition according to Item 2, wherein the brain tumor patient has an FGFR1 mutation in which the 546^{th} asparagine of FGFR1 is substituted with lysine or asparagine acid.
[4] The pharmaceutical composition according to Item [1], wherein the brain tumor patient has an FGFR1 mutation in which the 656^{th} lysine of FGFR1 is substituted with another amino acid.
[5] The pharmaceutical composition according to Item [4], wherein the brain tumor patient has an FGFR1 mutation in which the 656^{th} lysine of FGFR1 is substituted with glutamic acid, asparagine acid, asparagine, or methionine.
[6] The pharmaceutical composition according to Item [1], wherein the brain tumor patient has a mutation in which the 661^{st} arginine of FGFR1 is substituted with another amino acid.
[7] The pharmaceutical composition according to Item [6], wherein the brain tumor patient has an FGFR1 mutation in which the 661^{st} arginine of FGFR1 is substituted with proline.
[8] The pharmaceutical composition according to Item [1], wherein the brain tumor patient has an FGFR1-TACC1 fusion protein or FGFR1-TACC1 fusion gene.
[9] The pharmaceutical composition according to Item [1], wherein the brain tumor patient has at least one amino acid mutation selected from the group consisting of N546K, N546D, K656E, K656D, K656N, K656M, and R661P, or an FGFR1 mutation having an FGFR1-TACC1 fusion protein or FGFR1-TACC1 fusion gene.
[10] The pharmaceutical composition according to Item 1, wherein the brain tumor is glioblastoma, pilocytic astrocytoma, diffuse astrocytoma, anaplastic astrocytoma, gangliocytoma, ganglioglioma, anaplastic ganglioglioma, rosette-forming glioneuronal tumor, ependymoma, medulloblastoma, brainstem glioma, craniopharyngioma, anterior pituitary tumor, pheochromocytoma, chordoma, spongioblastoma, head and neck tumor, choroid plexus papilloma, choroid plexus carcinoma, oligodendroglioma, or anaplastic oligodendroglioma.
[11] A method for treating an FGFR1 mutant-positive brain tumor, comprising the step of administering an effective amount of (S)-1-(3-(4-amino-3-((3,5-dimethoxyphenyl)ethynyl)-1H-pyrazolo[3,4-d]pyrimidin-1-yl)pyrrolidin-1-yl)prop-2-en-1-one or a pharmaceutically acceptable salt thereof to an FGFR1 mutant-positive brain tumor patient.
[12] The method according to Item 11, comprising the steps of: detecting a mutation of an FGFR1 protein or FGFR1 gene from a sample derived from a brain tumor patient, and administering an effective amount of (S)-1-(3-(4-amino-3-((3,5-dimethoxyphenyl)ethynyl)-1H-pyrazolo[3,4-d]pyrimidin-1-yl)pyrrolidin-1-yl)prop-2-en-1-one or a pharmaceutically acceptable salt thereof to a patient from which the mutation of an FGFR1 protein or FGFR1 gene has been detected.
[13] The method according to Item 11, wherein the brain tumor patient has a mutation in which the 546^{th} asparagine of FGFR1 is substituted with another amino acid.
[14] The method according to Item 13, wherein the brain tumor patient has an FGFR1 mutation in which the 546^{th} asparagine of FGFR1 is substituted with lysine or asparagine acid.
[15] The method according to Item 11, wherein the brain tumor patient has a mutation in which the 656^{th} lysine of FGFR1 is substituted with another amino acid.
[16] The method according to Item 15, wherein the brain tumor patient has an FGFR1 mutation in which the 656^{th} lysine of FGFR1 is substituted with glutamic acid, asparagine acid, asparagine, or methionine.
[17] The method according to Item 11, wherein the FGFR1 mutant-positive brain tumor has a mutation in which the 661^{st} arginine of FGFR1 is substituted with another amino acid.
[18] The method according to Item 17, wherein the brain tumor patient has an FGFR1 mutant-positive brain tumor in which the 661^{st} arginine of FGFR1 is substituted with proline.
[19] The method according to Item [11], wherein the brain tumor patient has an FGFR1-TACC1 fusion protein or FGFR1-TACC1 fusion gene.
[20] The method according to Item [11], wherein the brain tumor patient has at least one amino acid mutation selected from the group consisting of N546K, N546D, K656E, K656D, K656N, K656M, and R661P, or an FGFR1 mutation having an FGFR1-TACC1 fusion protein or FGFR1-TACC1 fusion gene.
[21] The method according to Item 11, wherein the brain tumor is glioblastoma, pilocytic astrocytoma, diffuse astrocytoma, anaplastic astrocytoma, gangliocytoma, ganglioglioma, anaplastic ganglioglioma, rosette-forming glioneuronal tumor, ependymoma, medulloblastoma, brainstem glioma, craniopharyngioma, anterior pituitary tumor, pheochromocytoma, chordoma, spongioblastoma, head and neck tumor, choroid plexus papilloma, choroid plexus carcinoma, oligodendroglioma, or anaplastic oligodendroglioma.
[22] The method according to Item 11, wherein the administration is conducted every day or intermittently.
[23] The method according to Item 11, wherein the administration is conducted in an administration schedule of any one of the following (i) to (v):
(i) an administration schedule based on a 1-week cycle, in which (S)-1-(3-(4-amino-3-((3,5-dimethoxyphenyl)ethynyl)-1H-pyrazolo[3,4-d]pyrimidin-1-yl)pyrrolidin-1-yl)prop-2-en-1-one or a pharmaceutically acceptable salt thereof is administered at least twice every one to three days per cycle, and this cycle is performed once or repeated twice or more;
(ii) an administration schedule based on a 14-day cycle, in which Compound 1 or a pharmaceutically acceptable salt thereof is administered 4 to 7 times every one to three days per cycle (a dosing interval between a certain dosing date and the next dosing date of 1 to 3 days), and this cycle is performed once or repeated twice or more;
(iii) an administration schedule based on a 14-day cycle, in which, among 14 days contained in one cycle, Compound 1 or a pharmaceutically acceptable salt thereof is administered on Day 1, Day 4, Day 8, and Day 11;
(iv) an administration schedule based on a 14-day cycle, in which, among 14 days contained in one cycle, Compound 1 or a pharmaceutically acceptable salt thereof is administered on Day 1, Day 3, Day 5, Day 7, Day 9, Day 11, and Day 13; or
(v) an administration schedule based on a 14-day cycle, in which, among 14 days contained in one cycle, Compound 1 or a pharmaceutically acceptable salt thereof is administered on Day 1, Day 3, Day 5, Day 8, Day 10, and Day 12.

The present invention includes the following embodiments.
(S)-1-(3-(4-amino-3-((3,5-dimethoxyphenyl) ethynyl)-1H-pyrazolo[3,4-d]pyrimidin-1-yl)pyrrolidin-1-yl)prop-2-en-1-one or a pharmaceutically acceptable salt thereof for the treatment of an FGFR1-positive brain tumor.
Use of (S)-1-(3-(4-amino-3-((3,5-dimethoxyphenyl)ethynyl)-1H-pyrazolo[3,4-d]pyrimidin-1-yl)pyrrolidin-1-yl)prop-2-en-1-one or a pharmaceutically acceptable salt thereof for the treatment of an FGFR1-positive brain tumor.
Use of (S)-1-(3-(4-amino-3-((3,5-dimethoxyphenyl)ethynyl)-1H-pyrazolo[3,4-d]pyrimidin-1-yl)pyrrolidin-1-yl)prop-2-en-1-one or a pharmaceutically acceptable salt thereof in the manufacture of a pharmaceutical composition of an FGFR1-positive brain tumor.

### Advantageous Effects of Invention

### Description of Embodiments

The present invention can provide tumor therapy that exhibits excellent anti-tumor effects for FGFR1 mutant-positive brain tumors.

### DESCRIPTION OF EMBODIMENTS

The present invention relates to a pharmaceutical composition for treating a patient with an FGFR1 mutant-positive brain tumor, the composition comprising (S)-1-(3-(4-amino-3-((3,5-dimethoxyphenyl)ethynyl)-1H-pyrazolo[3,4-d]pyrimidin-1-yl)pyrrolidin-1-yl)prop-2-en-1-one or a pharmaceutically acceptable salt thereof as an active ingredient; and a method for treating an FGFR1 mutant-positive brain tumor using the pharmaceutical composition.

(S)-1-(3-(4-amino-3-((3,5-dimethoxyphenyl)ethynyl)-1H-pyrazolo[3,4-d]pyrimidin-1-yl)pyrrolidin-1-yl)prop-2-en-1-one (hereinbelow referred to as “Compound 1”) is a disubstituted benzene alkynyl compound having the following structure. Compound 1 is not particularly limited; however, it can be, for example, synthesized based on the production method described in WO2013/108809.

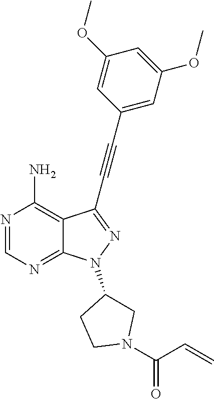 [SMILES: C=CC(=O)N1CC[C@H](n2nc(C#Cc3cc(OC)cc(OC)c3)c3c(N)ncnc32)C1]

In the present invention, Compound 1 can be used as is, or in the form of a pharmaceutically acceptable salt. The pharmaceutically acceptable salt of Compound 1 is not particularly limited, and examples include addition salts with inorganic acids such as hydrochloric acid, sulfuric acid, nitric acid, phosphoric acid, and hydrobromic acid, or with organic acids such as acetic acid, oxalic acid, citric acid, tartaric acid, maleic acid, benzene sulfonic acid, methanesulfonic acid, and toluene sulfonic acid; salts with alkali metals, such as potassium and sodium; salts with alkaline earth metals, such as calcium and magnesium; salts with organic bases, such as ammonium salts, ethylamine salts, and arginine salts; and the like.

In the present invention, “FGFR1” includes FGFR1 of humans or non-human mammals; human FGFR1 is preferred. The Gene ID number of human FGFR1 is 2260. Further, an FGFR1 protein comprises an isoform, which is a splicing variant thereof. Examples of a human-derived isoform include a polypeptide consisting of the amino acid sequence (SEQ ID NO: 1) encoded by the GenPept accession No. NP-075598.

In the present invention, “TACC1” includes TACC1 of a human or non-human mammal; human TACC1 is preferred. The Gene ID number of human TACC1 is 6867. Further, a TACC1 protein comprises an isoform, which is a splicing variant thereof. Examples of a human-derived isoform include a polypeptide comprising the amino acid sequence (SEQ ID NO: 2) encoded by the GenPept accession No. NP-001116296.

In the present invention, the “FGFR1 mutation” means an FGFR1 protein having an amino acid sequence in which at least one amino acid selected from the group consisting of the 546^{th} asparagine, 656^{th} lysine, and 661^{st} arginine in the amino acid sequence represented by SEQ ID NO: 1 is substituted with another amino acid, or an FGFR1 gene encoding the amino acid sequence; or an FGFR1-TACC1 fusion protein having an amino acid sequence in which FGFR1 is fused to TACC1, or an FGFR1-TACC1 fusion gene encoding the amino acid sequence. “Another amino acid” is selected from Ala, Cys, Asp, Glu, Phe, Gly, His, Ile, Lys, Leu, Met, Asn, Pro, Gln, Arg, Ser, Thr, Val, Trp, and Tyr; and means one of the 19 amino acids excluding the original amino acid.

As FGFR1 in which the 546^{th} asparagine is substituted with another amino acid, N546K in which the 546^{th} asparagine is substituted with lysine or N546D in which the 546^{th} asparagine is substituted with asparagine acid is preferred. As FGFR1 in which the 656^{th} lysine is substituted with another amino acid, K656E, K656D, K656N, and K656M in which the 656^{th} lysine is respectively substituted with glutamic acid, asparagine acid, asparagine, and methionine are preferred; and K656E or K656D is more preferred. As FGFR1 in which the 661^{st} arginine is substituted with another amino acid, R661P in which the 661^{st} arginine is substituted with proline is preferred.

The “FGFR1-TACC1 fusion protein” is a protein in which the N-terminal portion of FGFR1 protein is fused to the C-terminal portion of TACC1. “A protein in which the N-terminal portion of FGFR1 protein is fused to the C-terminal portion of TACC1” is a protein in which the N-terminal portion containing a kinase domain of FGFR1 protein is fused to the C-terminal portion containing part of or the entire transforming acidic coiled-coil (TACC) domain of TACC1, preferably a protein in which the N-terminal portion containing a kinase domain of FGFR1 protein is fused to the C-terminal portion containing the entire TACC domain of TACC1; more preferably a protein in which the N-terminal portion containing a kinase domain of FGFR1 protein is fused to the C-terminal portion containing the entire TACC domain of TACC1, said protein comprising a TSNQGLLE sequence (SEQ ID NO: 6) as the sequence of point of fusion; and even more preferably a protein (SEQ ID NO: 3) in which a protein having the amino acid sequence of positions 1 to 764 of FGFR1 represented by SEQ ID NO: 1 is fused to a protein having the amino acid sequence of positions 162 to 395 of TACC1 represented by SEQ ID NO: 2.

The “FGFR1-TACC1 fusion gene” means a gene encoding the amino acid sequence comprising an FGFR1-TACC1 fusion protein.

The FGFR1 mutation is preferably a protein comprising an amino acid sequence having at least one amino acid mutation selected from the group consisting of N546K, N546D, K656E, K656D, K656N, K656M, and R661P, or a gene encoding the amino acid sequence; or an FGFR1-TACC1 fusion protein or an FGFR1-TACC1 fusion gene. More preferably, the FGFR1 mutation is a protein comprising an amino acid sequence having at least one amino acid mutation selected from the group consisting of N546K, N546D, K656E, K656D, and R661P, or a gene encoding the amino acid sequence, or an FGFR1-TACC1 fusion protein or a FGFR1-TACC1 fusion gene.

Even if the position of the mutation in a certain FGFR1 isoform is different from the mutation position of the amino acid represented by SEQ ID NO: 1 due to the deletion or insertion of an amino acid, the mutation is considered to be the same as the mutation at the position corresponding to the amino acid represented by SEQ ID NO: 1. Accordingly, for example, the 656t lysine in FGFR1 represented by SEQ ID NO: 1 corresponds to the 687^{th} lysine in FGFR1 having the amino acid sequence (SEQ ID NO: 5) encoded by NP_001167538. Accordingly, “K656E” means that the 656^{th} lysine in FGFR1 represented by SEQ ID NO: 1 is substituted with glutamic acid; however, in FGFR1 comprising an amino acid sequence encoded by NP_001167538, the position corresponds to the 687^{th} amino acid. Thus, “K687E” in FGFR1 comprising an amino acid sequence represented by NP_001167538 corresponds to “K656E” in FGFR1 represented by SEQ ID NO: 1. To which position of the amino acid represented by SEQ ID NO: 1 a certain amino acid of a certain FGFR1 isoform corresponds can be confirmed, for example, by Multiple Alignment of BLAST.

The “protein in which the N-terminal portion containing a kinase domain of FGFR1 protein is fused to the C-terminal portion containing the entire transforming acidic coiled-coil (TACC) domain of TACC1, and said protein comprising a TSNQGLLE sequence as the sequence of point of fusion” is a protein obtained by fusing a protein in which the amino acid sequence at the C-terminus of the N-terminal portion containing the kinase domain of FGFR1 protein is TSNQ to a protein in which the amino acid sequence at the N-terminus of the C-terminal portion containing the entire TACC domain of TACC1 is GLLE.

Examples of the protein include a protein (SEQ ID NO: 3) in which a protein having the amino acid sequence of positions 1 to 764 of FGFR1 represented by SEQ ID NO: 1 is fused to a protein having the amino acid sequence of positions 162 to 395 of TACC1 represented by SEQ ID NO: 2; and a protein (SEQ ID NO: 4) in which a protein having the amino acid sequence of positions 1 to 673 of FGFR1 represented by GenPept accession No. NP-075594 is fused to a protein having the amino acid sequence of positions 162 to 395 of TACC1 represented by SEQ ID NO: 2.

In the present invention, “an FGFR1 mutant-positive brain tumor” is a brain tumor having an FGFR1 mutation. The target brain tumor is not particularly limited, and examples include brain tumors such as glioblastoma, pilocytic astrocytoma, diffuse astrocytoma, anaplastic astrocytoma, gangliocytoma, ganglioglioma, anaplastic ganglioglioma, rosette-forming glioneuronal tumor, ependymoma, medulloblastoma, brainstem glioma, craniopharyngioma, anterior pituitary tumor, pheochromocytoma, chordoma, spongioblastoma, head and neck tumor, choroid plexus papilloma, choroid plexus carcinoma, oligodendroglioma, or anaplastic oligodendroglioma; and preferably glioblastoma, pilocytic astrocytoma, rosette-forming glioneuronal tumor, ependymoma, or brainstem glioma. These brain tumors are not limited to primary tumors, but also include recurrent or metastatic tumors.

In the present invention, the FGFR1 mutation can be detected by a method that is well-known to those skilled in the art. Examples of the method of detecting a mutation of FGFR1 gene include conventionally known detection methods, such as Southern blotting, PCR, DNA microarray, and sequencing analysis. Examples of the method of detecting a mutation of FGFR1 protein include conventionally known detection methods, such as methods using an antibody that specifically binds to an FGFR1 mutation (ELISA, Western blotting, immunostaining, etc.), and mass spectral analysis. As the antibody that specifically binds to the FGFR1 mutation, a commercially available product can be used. Alternatively, the antibody can be produced by a conventionally known method.

In the present invention, the “sample” for detecting an FGFR1 mutation includes not only a biological sample (e.g., cells, tissues, organs, body fluids (blood, lymph fluid, and the like), digestive fluid, urine), but also a nucleic acid extract (e.g., genomic DNA extracts, mRNA extracts, cDNA preparation or cRNA preparation prepared from mRNA extracts, and the like) or a protein extract obtained from these biological samples. Further, the sample may be subjected to a formalin fixation treatment, an alcohol fixation treatment, a freezing treatment, or a paraffin embedding treatment. As the biological sample, a sample obtained from a living body can be used. The sample is preferably a sample derived from a malignant tumor patient, more preferably a sample that may contain an FGFR1 mutant protein or gene derived from tumor cells, even more preferably a sample that may contain FGFR1 mutant-positive brain tumor cells. The method for obtaining a biological sample can be suitably selected, depending on the type of biological sample. If mutant FGFR1 (protein or gene) can be detected from a brain tumor patient, the brain tumor can be determined to be an FGFR1 mutant-positive brain tumor.

The pharmaceutical composition of the present invention contains Compound 1 or a pharmaceutically acceptable salt thereof.

When Compound 1 or a pharmaceutically acceptable salt thereof is incorporated in a pharmaceutical preparation as an active ingredient, a pharmaceutical carrier can be added as necessary, thereby forming a suitable dosage form according to prevention and treatment purposes. Examples of the dosage form include oral preparations, injections, suppositories, ointments, patches, and the like. Of these, oral preparations are preferable. Examples of oral preparations include tablets, capsules, granules, powders, syrups, and the like, without any limitation. Such dosage forms can be formed by production methods conventionally known to persons skilled in the art. A suitable carrier, such as an excipient, diluent, bulking agent, or disintegrant, can be added, as necessary, to the pharmaceutical preparation or pharmaceutical composition according to the dosage form.

The amount of Compound 1 or a pharmaceutically acceptable salt thereof to be incorporated in each of such dosage unit forms depends on the condition of the patient to whom Compound 1 or its salt is administered, the dosage form, etc. In general, in the case of an oral preparation, an injection, and a suppository, the amount is preferably 0.05 to 1000 mg, 0.01 to 500 mg, and 1 to 1000 mg, respectively, per dosage unit form.

The dose of Compound 1 or a pharmaceutically acceptable salt thereof per day depends on the condition, body weight, age, gender, etc. of the patient, and cannot be generalized. For example, the dose of Compound 1 or a pharmaceutically acceptable salt thereof for an adult (body weight: 60 kg) per day is typically about 1 to 1000 mg, preferably about 10 to 500 mg, and more preferably about 10 to 300 mg.

When Compound 1 or a pharmaceutically acceptable salt thereof is administered every day, the dose of Compound 1 or a pharmaceutically acceptable salt thereof is, for example, about 1 to 200 mg; preferably 2 to 100 mg; more preferably 4 to 50 mg; even more preferably 4 to 20 mg; still more preferably 4, 8, 12, 16, or 20 mg; and yet more preferably 20 mg, per day.

When Compound 1 or a pharmaceutically acceptable salt thereof is administered intermittently, the dose of Compound 1 or a pharmaceutically acceptable salt thereof is, for example, about 2 to 1000 mg; preferably 10 to 500 mg; more preferably 20 to 200 mg, even more preferably 50 to 160 mg; still more preferably 52, 56, 60, 64, 68, 72, 76, 80, 88, 96, 100, 104, 108, 112, 116, 120, 124, 128, 132, 136, 140, 144, 148, 152, 156, or 160 mg; yet more preferably 60, 80, 100, 120, 140, or 160 mg; and particularly preferably 100, 120, 140, or 160 mg, per day.

Regarding the administration schedule, Compound 1 or its pharmaceutically acceptable salt can be administered every day, or intermittently.

In this specification, “administered every day” may be an administration schedule based on a cycle in which dosing is performed for 21 days every day (one cycle), and a period of drug holidays may be provided as each cycle ends.

In this specification, “administered intermittently” is not particularly limited, as long as the conditions of at least twice a week and a dosing interval of at least one day between dosings (the number of days between a certain dosing date and the next dosing date) are satisfied.

Examples include an administration schedule based on a 1-week cycle, in which Compound 1 or a pharmaceutically acceptable salt thereof is administered at least twice every one to three days per cycle (with a dosing interval between a certain dosing date and the next dosing date of 1 to 3 days), and this cycle is performed once or repeated twice or more; an administration schedule based on a 14-day cycle, in which Compound 1 or a pharmaceutically acceptable salt thereof is administered 4 to 7 times every one to three days per cycle (a dosing interval between a certain dosing date and the next dosing date of 1 to 3 days), and this cycle is performed once or repeated twice or more; an administration schedule based on a 14-day cycle, in which, among 14 days contained in one cycle, Compound 1 or a pharmaceutically acceptable salt thereof is administered on Day 1, Day 4, Day 8, and Day 11; an administration schedule based on a 14-day cycle, in which, among 14 days contained in one cycle, Compound 1 or a pharmaceutically acceptable salt thereof is administered on Day 1, Day 3, Day 5, Day 7, Day 9, Day 11, and Day 13; and an administration schedule based on a 14-day cycle, in which, among 14 days contained in one cycle, Compound 1 or a pharmaceutically acceptable salt thereof is administered on Day 1, Day 3, Day 5, Day 8, Day 10, and Day 12.

The present invention also provides a method of treating an FGFR1-positive brain tumor comprising the step of administering an effective amount of Compound 1 or a pharmaceutically acceptable salt thereof to a patient of an FGFR1-positive brain tumor.

The present invention also provides a method of treating an FGFR1-positive brain tumor, comprising the following steps (1) and (2):

(1) detecting a mutation of FGFR1 protein or FGFR1 gene from a sample derived from a patient; and
(2) administering an effective amount of Compound 1 or a pharmaceutically acceptable salt thereof to the patient to whom the mutation of FGFR1 protein or FGFR1 gene has been detected in step (1) above.

In the above therapeutic method, it is assumed that a chemotherapy in which an effective amount of Compound 1 or a pharmaceutically acceptable salt thereof is administered has a sufficient therapeutic effect on a patient from whom the mutation of FGFR1 protein or FGFR1 gene is detected. Here, “the therapeutic effect” can be evaluated by tumor shrinkage effects, relapse- or metastasis-suppressing effects, life-prolonging effects, and the revised Response Evaluation Criteria In Solid Tumors (RECIST) guidelines. The therapeutic effect can also be estimated by the degree of function inhibitory activity of FGFR1 (e.g., inhibitory activity using FGFR1 phosphorylation as an index).

### EXAMPLES

The present invention is detailed below with reference to Examples; however, the present invention is not limited thereto. The present invention is fully described below by way of Examples; however, it can be understood that various changes or modifications by a skilled artisan are possible. Therefore, such changes or modifications are included in the present invention as long as they do not depart from the scope of the invention.

### Example 1: Evaluation of Inhibitory Activity of Compound 1 Against Single-Amino-Acid Substitution Mutant of FGFR1 or TACC1 Fusion In Vitro

### 1-1 Construction of FGFR1 Point Mutant or TACC1 Fusion Expression Vector

As an FGFR1 vector, FGFR1 (NM_023110) Human Tagged ORF Clone (FGFR1 wild-type (WT) expression vector) purchased from ORIGENE was used. Vectors for expressing respective mutants (N546K, N546D, K656E, K656D, K656N, K656M, and R661P) were constructed using the above vector as a template, and using PrimeSTAR Max DNA Polymerase (Takara Bio Inc.).

Further, vectors for expressing an FGFR1-TACC1 fusion (a protein having an amino acid sequence represented by SEQ ID NO: 3) were constructed using the above vector and TACC1 (NM_001122824) Human Tagged ORF Clone (TACC1 wild expression vector) purchased from ORIGENE as templates, and using an In-Fusion HD Cloning Kit (Takara Bio Inc.).

### 1-2 Measurement of Inhibitory Activity of FGFR1 Using FGFR1 Phosphorylation as an Index

Human embryonic kidney cells (HEK293T) were cultured in a DMEM containing 10% fetal bovine serum. After the cells were collected by a normal method, they were suspended in a DMEM containing 10% fetal bovine serum. According to the lipotransfection method using a Lipofectamine 3000 reagent (Thermo Fisher Scientific), the FGFR1 wild-type, point mutant, or TACC1 fusion expression vectors were individually introduced into the cells. The cells were then seeded at 1.5×10⁴ cells/100 μL per well in a 96-well plate.

As a drug solution, a vehicle (DMSO) group and a diluent series (Compound 1: diluent series having 9 concentrations, including 3000 nM as the maximum final concentration, 1000, 300, 100, 30, 10, 3, 1, and 0.3 nM; AZD4547, BGJ398, and JNJ42756493: diluent series having 10 concentrations, including 10000 nM as the maximum final concentration, 3000, 1000, 300, 100, 30, 10, 3, 1, and 0.3 nM) of Compound 1, AZD4547, BGJ398 (Chemietek), and JNJ42756493 (Sundia) were prepared. The seeded cells were incubated at 37° C., 5% CO₂ for 24 hours; and then 11 μL of medium containing a drug solution was added thereto, followed by incubation for another one hour.

Functional inhibition against the autophosphorylation ability of FGFR1 was evaluated using a Human Phospho-FGFR1 DuoSet IC ELISA (R&D Systems). A protease inhibitor (Roche) and a phosphatase inhibitor (Roche) were added to a cell lysate attached to the kit, and the cells were dissolved using them. An experiment was conducted according to the protocol of the kit. Colorimetric quantification was performed on each well using a plate reader (Spectra MAX384, Molecular Devices). The relative FGFR1 phosphorylation percentage in a drug solution-added well was calculated according to the following formula, using the control group as 100%. The experiment was conducted in duplicate (21·2 wells per treatment group), and the average of the data of the two wells was used for the analysis.

Relative FGFR1 phosphorylation percentage (%)=(signal amount in the drug solution-added well)/(signal amount in the control group)×100

The IC₅₀ value (50% inhibition concentration) was calculated as the concentration at which 50% of inhibition was achieved relative to the control group.

In 293T cell lines in which the FGFR1 wild-type, a single-amino-acid substitution mutant, or a TACC1 fusion was expressed, Compound 1 showed the following inhibitory activity (Tables 1 and 2).

TABLE 1

| | Inhibition of FGFR1 phosphorylation IC₅₀ (nM) | | | | |
|---|---|---|---|---|---|
| | WT | N546K | N546D | K656E | K656D |
| Compound 1 | 3.7 | 40.4 | 27.8 | 21.9 | 16.0 |
| AZD4547 | 15.9 | 357.6 | 831.7 | 221.8 | 172.3 |
| BGJ398 | 9.9 | 89.3 | 299.7 | 70.6 | 36.0 |
| JNJ42756493 | 5.1 | 55.5 | 229.1 | 30.8 | 26.8 |

TABLE 2

| | Inhibition of FGFR1 phosphorylation IC₅₀ (nM) | | | |
|---|---|---|---|---|
| | K656N | K656M | R661P | FGFR1-TACC1 |
| Compound 1 | 8.0 | 25.6 | 3.1 | 4.5 |
| AZD4547 | 46.5 | 296.0 | 12.6 | 13.6 |
| BGJ398 | 16.8 | 122.6 | 6.3 | 8.5 |
| JNJ42756493 | 8.4 | 58.5 | 3.6 | 5.2 |

P20-036WO_PCT_FGFR1 mutant-positive brain_20200217_171826_4.txt

1. A pharmaceutical composition for treating an FGFR1 mutant-positive brain tumor patient, comprising (S)-1-(3-(4-amino-3-((3,5-dimethoxyphenyl)ethynyl)-1H-pyrazolo[3,4-d]pyrimidin-1-yl)pyrrolidin-1-yl)prop-2-en-1-one or a pharmaceutically acceptable salt thereof as an active ingredient.

2. The pharmaceutical composition according to claim 1, wherein the brain tumor patient has a mutation in which the 546^{th} asparagine of FGFR1 is substituted with another amino acid.

3. The pharmaceutical composition according to claim 2, wherein the brain tumor patient has an FGFR1 mutation in which the 546^{th} asparagine of FGFR1 is substituted with lysine or asparagine acid.

4. The pharmaceutical composition according to claim 1, wherein the brain tumor patient has an FGFR1 mutation in which the 656^{th} lysine of FGFR1 is substituted with another amino acid.

5. The pharmaceutical composition according to claim 4, wherein the brain tumor patient has an FGFR1 mutation in which the 656^{th} lysine of FGFR1 is substituted with glutamic acid, asparagine acid, asparagine, or methionine.

6. The pharmaceutical composition according to claim 1, wherein the brain tumor patient has a mutation in which the 661^{st} arginine of FGFR1 is substituted with another amino acid.

7. The pharmaceutical composition according to claim 6, wherein the brain tumor patient has an FGFR1 mutation in which the 661^{st} arginine of FGFR1 is substituted with proline.

8. The pharmaceutical composition according to claim 1, wherein the brain tumor patient has an FGFR1-TACC1 fusion protein or FGFR1-TACC1 fusion gene.

9. The pharmaceutical composition according to claim 1, wherein the brain tumor patient has at least one amino acid mutation selected from the group consisting of N546K, N546D, K656E, K656D, K656N, K656M, and R661P, or an FGFR1 mutation having an FGFR1-TACC1 fusion protein or FGFR1-TACC1 fusion gene.

10. The pharmaceutical composition according to claim 1, wherein the brain tumor is glioblastoma, pilocytic astrocytoma, diffuse astrocytoma, anaplastic astrocytoma, gangliocytoma, ganglioglioma, anaplastic ganglioglioma, rosette-forming glioneuronal tumor, ependymoma, medulloblastoma, brainstem glioma, craniopharyngioma, anterior pituitary tumor, pheochromocytoma, chordoma, spongioblastoma, head and neck tumor, choroid plexus papilloma, choroid plexus carcinoma, oligodendroglioma, or anaplastic oligodendroglioma.

11. A method for treating an FGFR1 mutant-positive brain tumor, comprising the step of administering an effective amount of (S)-1-(3-(4-amino-3-((3,5-dimethoxyphenyl) ethynyl)-1H-pyrazolo[3,4-d]pyrimidin-1-yl)pyrrolidin-1-yl)prop-2-en-1-one or a pharmaceutically acceptable salt thereof to an FGFR1 mutant-positive brain tumor patient.

12. The method according to claim 11, comprising the steps of:
detecting a mutation of an FGFR1 protein or FGFR1 gene from a sample derived from a brain tumor patient, and administering an effective amount of (S)-1-(3-(4-amino-3-((3,5-dimethoxyphenyl)ethynyl)-1H-pyrazolo[3,4-d]pyrimidin-1-yl)pyrrolidin-1-yl)prop-2-en-1-one or a pharmaceutically acceptable salt thereof to a patient from which the mutation of an FGFR1 protein or FGFR1 gene has been detected.

13. The method according to claim 11, wherein the brain tumor patient has a mutation in which the 546^{th} asparagine of FGFR1 is substituted with another amino acid.

14. The method according to claim 13, wherein the brain tumor patient has an FGFR1 mutation in which the 546^{th} asparagine of FGFR1 is substituted with lysine or asparagine acid.

15. The method according to claim 11, wherein the brain tumor patient has a mutation in which the 656^{th} lysine of FGFR1 is substituted with another amino acid.

16. The method according to claim 15, wherein the brain tumor patient has an FGFR1 mutation in which the 656^{th} lysine of FGFR1 is substituted with glutamic acid, asparagine acid, asparagine, or methionine.

17. The method according to claim 11, wherein the FGFR1 mutant-positive brain tumor has a mutation in which the 661^{st} arginine of FGFR1 is substituted with another amino acid.

18. The method according to claim 17, wherein the brain tumor patient has an FGFR1 mutant-positive brain tumor in which the 661^{st} arginine of FGFR1 is substituted with proline.

19. The method according to claim 11, wherein the brain tumor patient has an FGFR1-TACC1 fusion protein or FGFR1-TACC1 fusion gene.

20. The method according to claim 11, wherein the brain tumor patient has at least one amino acid mutation selected from the group consisting of N546K, N546D, K656E, K656D, K656N, K656M, and R661P, or an FGFR1 mutation having an FGFR1-TACC1 fusion protein or FGFR1-TACC1 fusion gene.

21. The method according to claim 11, wherein the brain tumor is glioblastoma, pilocytic astrocytoma, diffuse astrocytoma, anaplastic astrocytoma, gangliocytoma, ganglioglioma, anaplastic ganglioglioma, rosette-forming glioneuronal tumor, ependymoma, medulloblastoma, brainstem glioma, craniopharyngioma, anterior pituitary tumor, pheochromocytoma, chordoma, spongioblastoma, head and neck tumor, choroid plexus papilloma, choroid plexus carcinoma, oligodendroglioma, or anaplastic oligodendroglioma.

22. The method according to claim 11, wherein the administration is conducted every day or intermittently.

23. The method according to claim 11, wherein the administration is conducted in an administration schedule of any one of the following (i) to (v):
(i) an administration schedule based on a 1-week cycle, in which (S)-1-(3-(4-amino-3-((3,5-dimethoxyphenyl)ethynyl)-1H-pyrazolo[3,4-d]pyrimidin-1-yl)pyrrolidin-1-yl)prop-2-en-1-one or a pharmaceutically acceptable salt thereof is administered at least twice every one to three days per cycle, and this cycle is performed once or repeated twice or more;
(ii) an administration schedule based on a 14-day cycle, in which Compound 1 or a pharmaceutically acceptable salt thereof is administered 4 to 7 times every one to three days per cycle (a dosing interval between a certain dosing date and the next dosing date of 1 to 3 days), and this cycle is performed once or repeated twice or more;
(iii) an administration schedule based on a 14-day cycle, in which, among 14 days contained in one cycle, Compound 1 or a pharmaceutically acceptable salt thereof is administered on Day 1, Day 4, Day 8, and Day 11;
(iv) an administration schedule based on a 14-day cycle, in which, among 14 days contained in one cycle, Compound 1 or a pharmaceutically acceptable salt thereof is administered on Day 1, Day 3, Day 5, Day 7, Day 9, Day 11, and Day 13; or
(v) an administration schedule based on a 14-day cycle, in which, among 14 days contained in one cycle, Compound 1 or a pharmaceutically acceptable salt thereof is administered on Day 1, Day 3, Day 5, Day 8, Day 10, and Day 12.


SEQUENCE LISTING (6 sequences)

SEQ ID NO: 1 - protein, 822 aa, Homo sapiens [sequence omitted]

SEQ ID NO: 2 - protein, 395 aa, Homo sapiens
MAFSPWQILSPVQWAKWTWSAVRGGAAGEDEAGGPEGDPEEEDSQAETKSLSFRSGCKVKKHETQSLALDACSRDEGAVISQISDISNRDGHATDEEKLASTSCGQKSAGAEVKGEPEEDLEYFECSNVPVSTINHAFSSSEAGIEKETCQKMEEDGSTVLGLLESSAEKAPVSVSCGGESPLDGICLSESDKTAVLTLIREEIITKEIEANEWKKKYEETRQEVLEMRKIVAEYEKTIAQMIEDEQRTSMTSQKSFQQLTMEKEQALADLNSVERSLSDLFRRYENLKGVLEGFKKNEEALKKCAQDYLARVKQEEQRYQALKIHAEEKLDKANEEIAQVRTKAKAESAALHAGLRKEQMKVESLERALQQKNQEIEELTKICDELIAKLGKTD

SEQ ID NO: 3 - protein, 998 aa, Homo sapiens
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNRMPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQGLLESSAEKAPVSVSCGGESPLDGICLSESDKTAVLTLIREEIITKEIEANEWKKKYEETRQEVLEMRKIVAEYEKTIAQMIEDEQRTSMTSQKSFQQLTMEKEQALADLNSVERSLSDLFRRYENLKGVLEGFKKNEEALKKCAQDYLARVKQEEQRYQALKIHAEEKLDKANEEIAQVRTKAKAESAALHAGLRKEQMKVESLERALQQKNQEIEELTKICDELIAKLGKTD

SEQ ID NO: 4 - protein, 907 aa, Homo sapiens
MWSWKCLLFWAVLVTATLCTARPSPTLPEQDALPSSEDDDDDDDSSSEEKETDNTKPNPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQGLLESSAEKAPVSVSCGGESPLDGICLSESDKTAVLTLIREEIITKEIEANEWKKKYEETRQEVLEMRKIVAEYEKTIAQMIEDEQRTSMTSQKSFQQLTMEKEQALADLNSVERSLSDLFRRYENLKGVLEGFKKNEEALKKCAQDYLARVKQEEQRYQALKIHAEEKLDKANEEIAQVRTKAKAESAALHAGLRKEQMKVESLERALQQKNQEIEELTKICDELIAKLGKTD

SEQ ID NO: 5 - protein, 853 aa, Homo sapiens
MEARVSLKRRIELTVEYPWRCGALSPTSNCRTGMWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 6 - protein, 8 aa, Homo sapiens
TSNQGLLE